(19) United States
(12) Patent Application Publication
(10) Pub. No.: US 2017/0181883 A1
(43) Pub. Date: Jun. 29, 2017

(54) SYSTEMS AND METHODS FOR EFFECTIVE POSTURE OF A SUBJECT

(71) Applicant: Innovative Intelligence, LLC, Lumberton, NJ (US)

(72) Inventors: Manoj B. Patel, Lumberton, NJ (US); Sutchin R. Patel, Lake Forest, IL (US); Robert Dickie, King City (CA)

(21) Appl. No.: 14/980,808

(22) Filed: Dec. 28, 2015

(51) Int. Cl. A61F 5/37; A61G 7/057; A61G 7/015; A61F 5/34

(52) U.S. Cl. CPC A61F 5/3784; A61F 5/34; A61G 7/05707; A61G 7/015

(57) ABSTRACT

Systems and methods for effecting posture of a subject who is to be positioned on a support surface having an inclinable portion such that the subject's thorax is inclinable relative to the subject's legs, are provided. An example system includes a detachedly coupleable thoracic harness having at least an inner surface and an outer surface. The inner surface being contoured to fit over shoulders of the subject. The outer surface having a fastening surface that releasable attaches to a corresponding fastening surface disposed on the inclinable portion of the support surface.

### TECHNICAL FIELD

This disclosure relates to a medical device, and more particularly to a medical device for effecting posture of a subject.

### BACKGROUND

Patients who are on ventilators are at an increased risk of developing Ventilator-Associated Pneumonia (VAP), especially patients with a respiratory illnesses such as chronic obstructive pulmonary disease. VAP is defined as a lung infection that occurs after at least 48 hours of intubation and mechanical ventilation. VAP can increase a patent's mortality rate, length of stay in hospital, and costs. VAP is the leading cause of death due to nosocomial infections and the second most common nosocomial infection in the United States. Thus the prevention of VAP is a priority.

In 2008, the Centers for Disease Control recommended a head of bed (HOB) elevation of between 30 to 45 degrees to prevent VAP, maximize ventilation, and improve total lung volume. However, in present practice this HOB elevation has been often associated with the patient “sinking” to the foot of the bed, which can cause the neck of a patient to flex. A flexed neck can lead to an impaired airway. Flexion of the lumbar spine can shift the peritoneal contents upward, raising the diaphragm, which can result in lowering the Functional Residual Capacity (FRC) of the lungs and increasing the shunt with resulting hypoxia.

Although, a proper HOB elevation and upright position may reduce VAP, increasing HOB conflicts with present guidelines for reducing decubitus pressure ulcers. Peak sacral interface pressures increase with large increases in HOB elevation, especially if HOB angle is greater than 45 degrees. Normally, capillary pressure in buttock skin ranges from 10-30 mmHg. When this pressure is exceeded, oxygen delivery to this region is compromised, thus leading to lower blood flow and increased tissue ischemia, which can result in the formation of pressure ulcers. HOB angles of about 30, 45, 60, and 75 degrees can have increased peak sacral interface pressures, but only when the HOB angle is above 45 degrees does the peak sacral interface pressure exceed 32 mmHg, which is the critical value for the formation of decubitus ulcers.

Therefore, improved methods and devices for effecting posture of a subject are needed, which can, for example, provide an elevated HOB while reducing the likelihood of decubitus pressure ulcers being formed.

### SUMMARY

Provided are systems and methods for effecting posture of a subject who is to be positioned on a support surface having an inclinable portion such that the subject's thorax is inclinable relative to the subject's legs.

An example system includes a detachedly coupleable thoracic harness having at least an inner surface and an outer surface. The inner surface being contoured to fit over shoulders of the subject. The outer surface having a fastening surface that releasably attaches to a corresponding fastening surface disposed on the inclinable portion of the support surface.

The details of one or more embodiments of the disclosure are set forth in the accompanying drawings and the description below. Other features, objects, and advantages of the disclosure will be apparent from the description, drawings, and from the claims.

### DESCRIPTION OF DRAWINGS

To facilitate an understanding of and for the purpose of illustrating the present disclosure, exemplary features and implementations are disclosed in the accompanying drawings, it being understood, however, that the present disclosure is not limited to the precise arrangements and instrumentalities shown, and wherein similar reference characters denote similar elements throughout the several views, and wherein:

FIG. 1 is a perspective view of an example system for effecting posture of a subject in accordance with the present disclosure, the view includes a person, a support surface, a support pad, a lumbar support, a femoral-gluteal pad, and a detachedly coupleable thoracic harness embodied as a vest;

FIG. 2 is a perspective view of an example system for effecting posture of a subject in accordance with the present disclosure, the view includes a person, a support surface, a support pad, a lumbar support, a femoral-gluteal pad, and a detachedly coupleable thoracic harness embodied as a pair of shoulder guards;

FIG. 3 is a perspective view of an example system for effecting posture of a subject in accordance with the present disclosure, the view includes a support surface, a support pad, a lumbar support, and a femoral-gluteal pad;

FIG. 4 is a perspective view of an example system for effecting posture of a subject in accordance with the present disclosure, the view includes a support surface, a support pad, a lumbar support, and a femoral-gluteal pad;

FIG. 5 is a top view of a an example system for effecting posture of a subject in accordance with the present disclosure, the view includes a person, a support surface, a support pad, a lumbar support, and a femoral-gluteal pad;

FIG. 6 is a side view of an example system for effecting posture of a subject in accordance with the present disclosure, the view includes a person, a support surface, a support pad, a lumbar support, and a femoral-gluteal pad;

FIG. 7 is an exploded view of the femoral-gluteal pad shown in FIG. 6;

FIG. 8 is a top view of an example system for effecting posture of a subject in accordance with the present disclosure, the view includes a person, a support surface, a support pad, a lumbar support, a femoral-gluteal pad, and a detachedly coupleable thoracic harness embodied as a vest;

FIG. 9 is a top view of an example system for effecting posture of a subject in accordance with the present disclosure, the view includes a person, a support surface, a support pad, a lumbar support, a femoral-gluteal pad, and a detachedly coupleable thoracic harness embodied as a pair of shoulder guards;

FIG. 10 is a side view of the system shown in FIG. 8;

FIG. 11 is a side view of the system shown in FIG. 9;

FIG. 12 is a top view of an example support pad in accordance with the present disclosure;

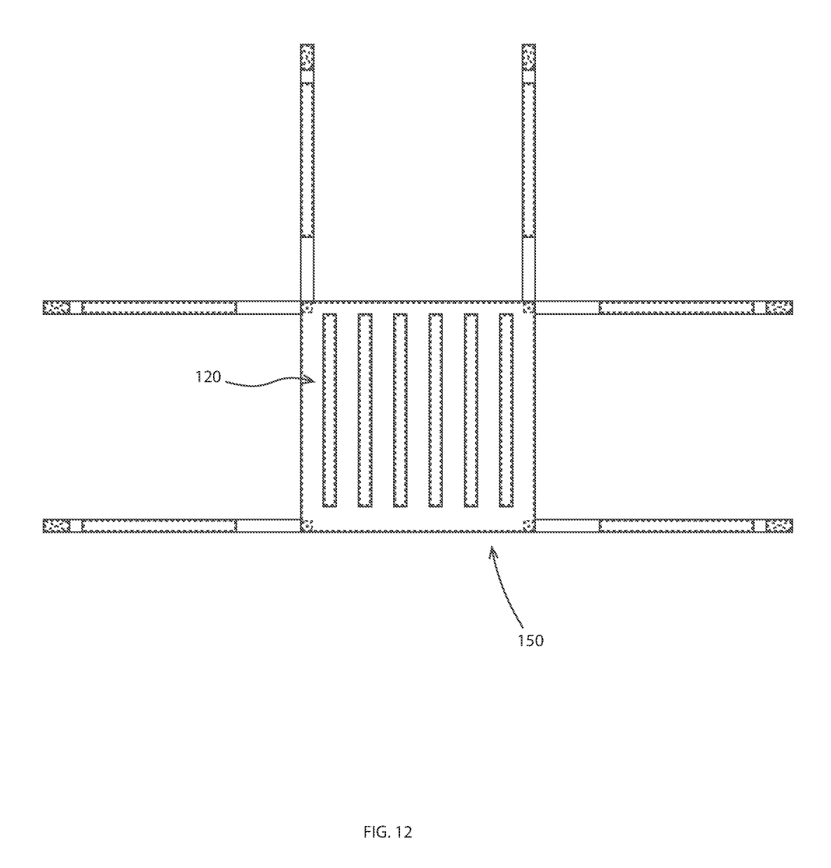

FIG. 13 is an outer surface view of an example detachedly coupleable thoracic harness in accordance with the present disclosure, in this view the detachedly coupleable thoracic harness embodies a vest;

FIG. 14 is an inner surface view of the detachedly coupleable thoracic harness of FIG. 13;

FIG. 15 is a perspective view of an example detachedly coupleable thoracic harness in accordance with the present disclosure, in this view the detachedly coupleable thoracic harness embodies a shoulder guard;

FIG. 16 is a side view of the detachedly coupleable thoracic harness shown in FIG. 15;

FIG. 17 is a top view of the detachedly coupleable thoracic harness shown in FIG. 15 and FIG. 16;

FIG. 18 is a perspective view of an example detachedly coupleable thoracic harness in accordance with the present disclosure, in this view the detachedly coupleable thoracic harness embodies a pair of shoulder guards;

FIG. 19 is a top view of an example support pad in accordance with the present disclosure;

FIG. 20 is a top view of an example support pad in accordance with the present disclosure;

FIG. 21 is an exploded cross-sectional view of a portion of the support pad shown in FIG. 20;

FIG. 22 is an inner surface view of an example detachedly coupleable thoracic harness in accordance with the present disclosure, in this view the detachedly coupleable thoracic harness embodies a vest;

FIG. 23 is an outer surface view of the detachedly coupleable thoracic harness of FIG. 22;

FIG. 24 is an outer surface view of an example detachedly coupleable thoracic harness in accordance with the present disclosure, in this view the detachedly coupleable thoracic harness embodies a vest;

FIG. 25 is an inner surface view of the detachedly coupleable thoracic harness of FIG. 24;

FIG. 26 is a partial top view of an example femoral-gluteal pad in accordance with the present disclosure;

FIG. 27 is a cross-sectional view of an example femoral-gluteal pad in accordance with the present disclosure; and

FIG. 28 is a cross-sectional view of an example femoral-gluteal pad in accordance with the present disclosure.

### DETAILED DESCRIPTION

The following is a description of several illustrations of the subject matter of Applicant's invention. Certain terminology is used herein for convenience only and is not to be taken as a limitation on the present invention. In the drawings, the same reference numbers are employed for designating the same elements throughout the several figures. A number of examples are provided, nevertheless, it will be understood that various modifications can be made without departing from the spirit and scope of the disclosure herein. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed.

The present invention now will be described more fully hereinafter with reference to specific embodiments of the invention. Indeed, the invention can be embodied in many different forms and should not be construed as limited to the embodiments set forth herein; rather, these embodiments are provided so that this disclosure will satisfy applicable legal requirements.

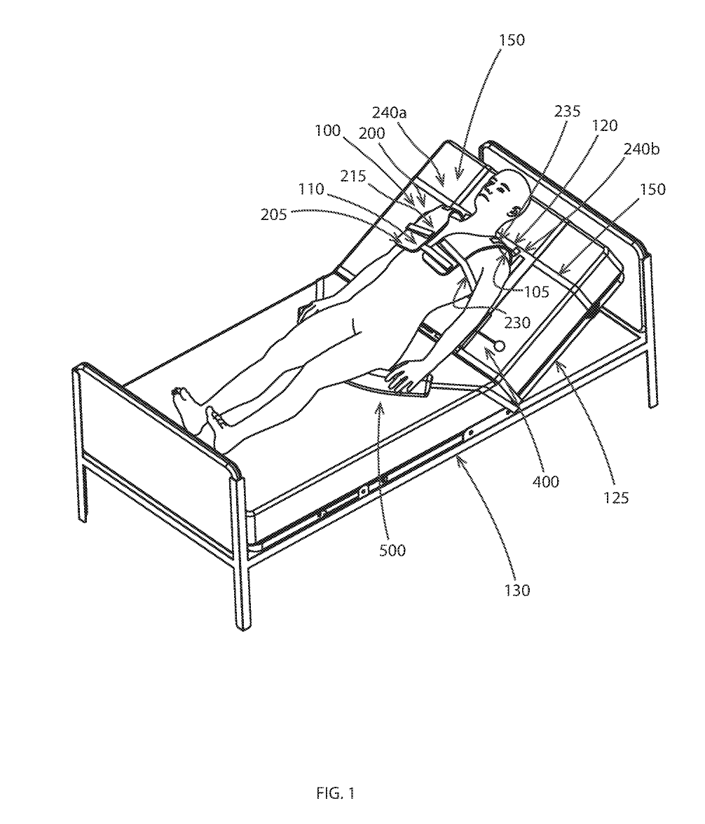

FIG. 1 shows an example system for effecting posture of a subject. The example system includes a detachedly coupleable thoracic harness 100 which can be used to effect posture of a subject who is to be positioned on a support surface 130 having an inclinable portion 125 such that the subject's thorax is inclinable relative to the subject's legs. Thus, the subject's lower body may remain in a horizontal orientation while the subject's upper body is inclined relative to the subject's lower body, for example.

Example support surfaces 130 include folding beds, reclining chairs, reclining wheelchairs, and other suitable support devices having at least one inclinable portion. In the context of FIG. 1, the support surface 130 takes the form of a folding bed having a head portion and a foot portion. The head portion can be pivotally connected to the foot portion. The head portion can bend and/or fold in reference to the foot portion such that an angle is created between the head portion and the foot portion. In certain embodiments the inclinable portion 125 is the only portion of support surface 130 which is inclinable.

An example detachedly coupleable thoracic harness 100 includes an inner surface 105 contoured to fit over the shoulders of a subject and includes an outer surface 110 having a fastening surface 115 that releasably attaches to a corresponding fastening surface 120 disposed on the inclinable portion 125 of the support surface 130. In use, the detachedly coupleable thoracic harness 100 allows one to maintain the subject at a desired HOB elevation, for example about 30-45 degrees, or over about 45 degrees, while limiting sliding and slouching of the subject in reference to the inclinable portion 125.

In accordance with the present disclosure, the fastening surface 115 of the detachedly coupleable thoracic harness 100 and the corresponding fastening surface 120 include, for example, reciprocal parts of a hook-and-loop fastener system such as Velcro®, sold by Velcro Industries B.V. (Curacao, Netherlands), recloseable fasteners such as Dual Lock®, sold by 3M (Saint Paul, Minn.), and other fasters such as reusable adhesives, suctions, magnets, tape, and flexible magnet tape, for example.

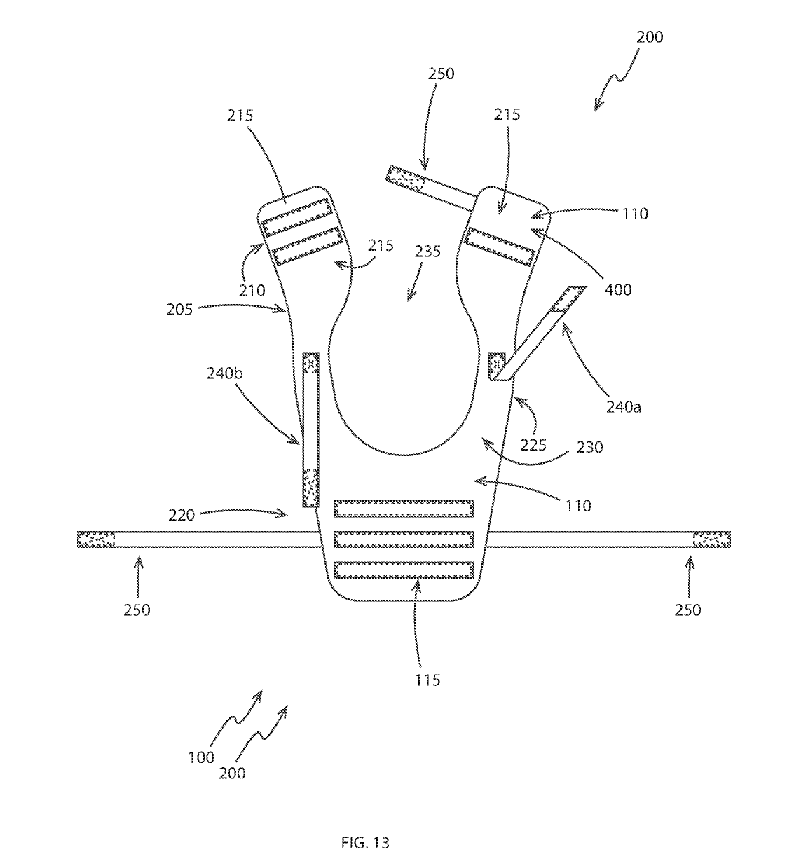

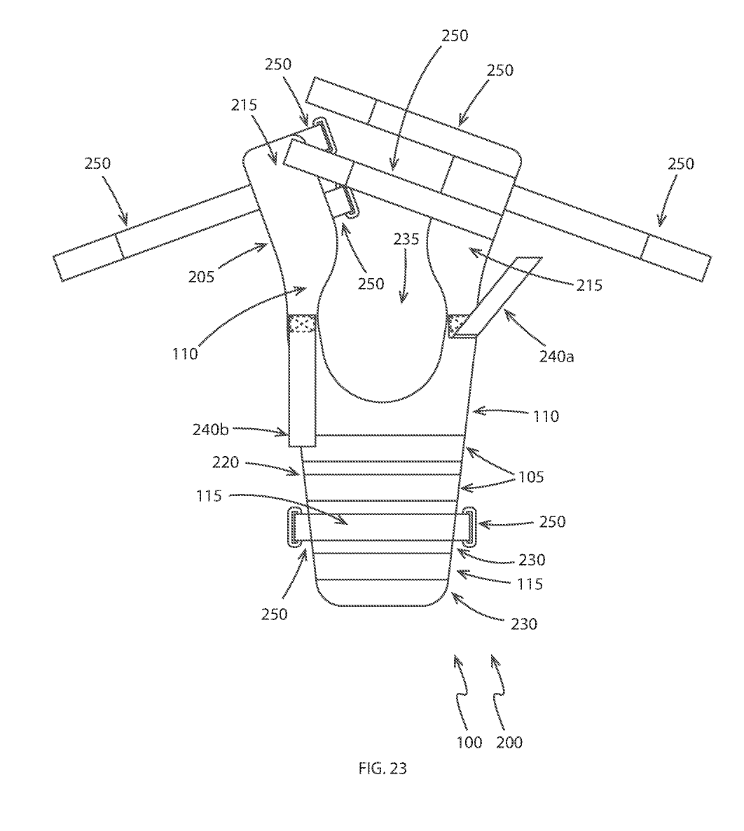

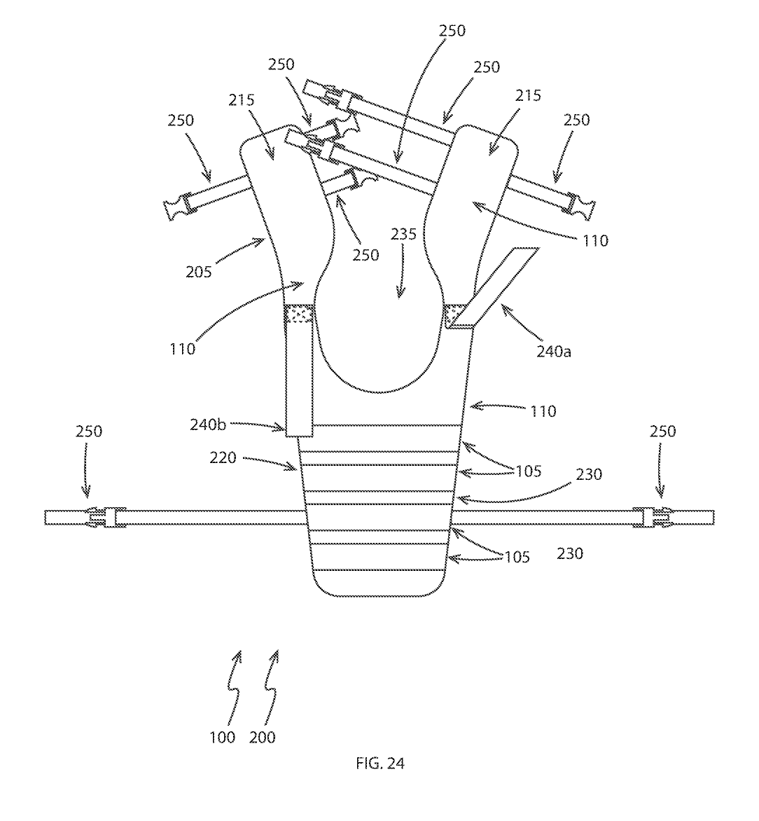

In the context of a hook- and loop-fastener system, a plurality of hook portions can be disposed on the outer surface 110 of the detachedly coupleable thoracic harness 100 and a plurality of loop portions can be disposed on the inclinable portion 125 of the support surface 130 to releasably fasten the outer surface 110 of the detachedly coupleable thoracic harness 100 to the inclinable portion 125 of the support surface 130. In certain embodiments, the plurality of hook portions can be disposed symmetrically down the outer surface 110 of the detachedly coupleable thoracic harness 110. In certain embodiments, the plurality of hook portions can be disposed on one or more portions of the outer surface 110 and/or disposed on the entire outer surface 110. As shown in FIGS. 13, 23 and 24, the plurality of hook portions can be disposed as one or more horizontal strips. In certain embodiments, the plurality of hook portions can be disposed as a plurality of patches. The patches can take the form of various suitable shapes, including, for example, a circles and triangles. The patches can be similarly disposed symmetrically about the outer surface 110.

Similar to the plurality of hook portions, the plurality of loop portions can be disposed on the inclinable portion 125 of the support surface 130 in various suitable ways. For example, the plurality of loop portions can be disposed as a plurality of parallel strips that are placed along the inclinable portion 125 of the support surface 130. In certain embodiments, the plurality of loop portions can be disposed on the inclinable portion 125 of the support surface 130 via a structure coupled to the support surface 130, such as a support surface pad 150. In some embodiments, the support surface pad 150 can be a permanent fixture of the support surface 130. In other embodiments, the support surface pad 150 can be a removable and/or reusable element. The plurality of loops can cover the entire inclinable portion 125 of the support surface 130.

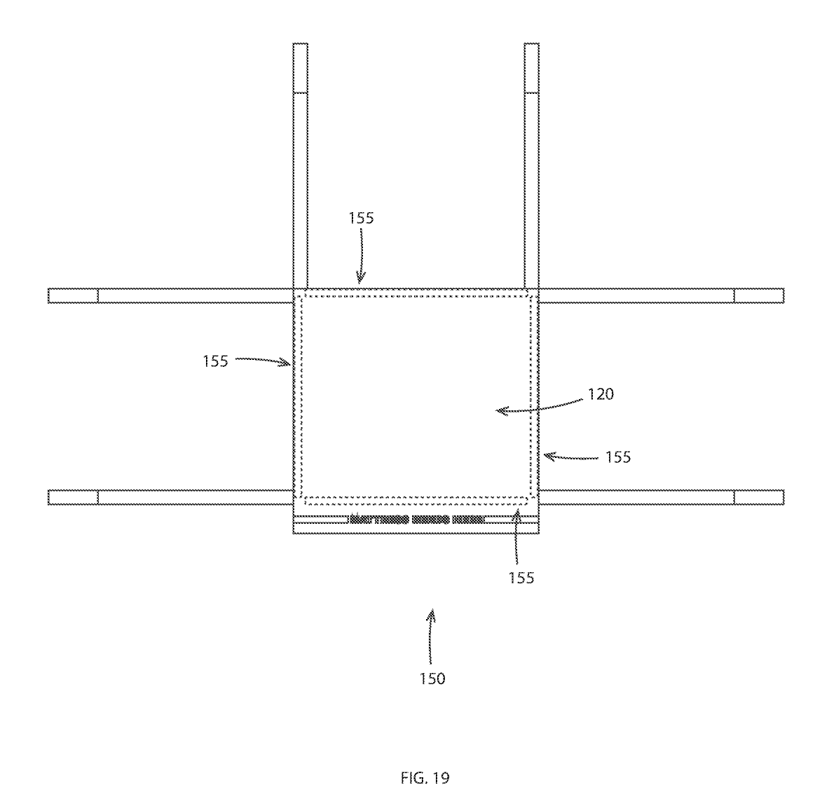

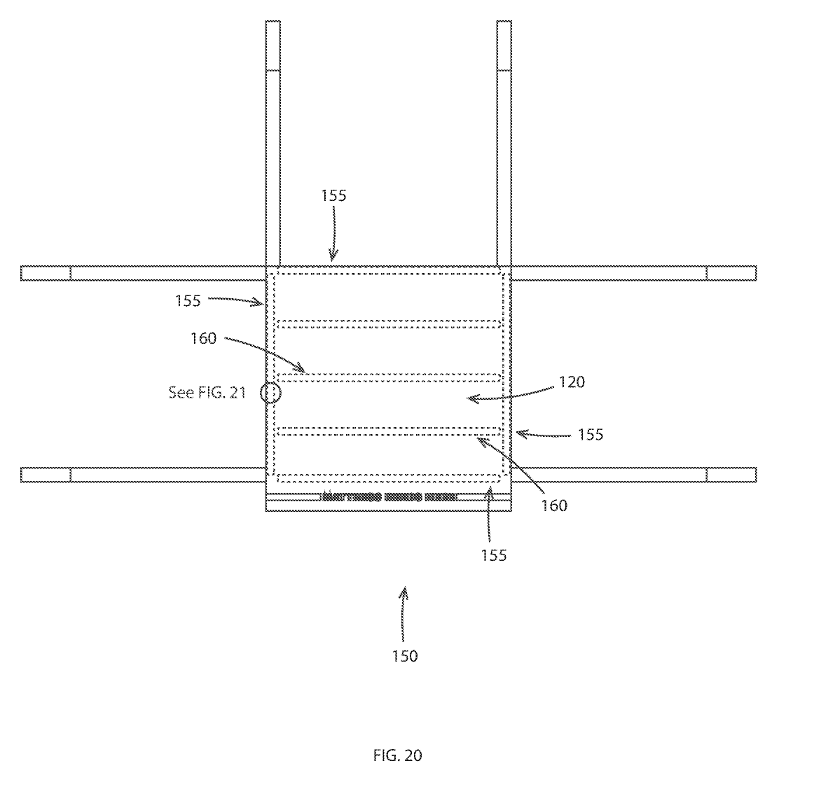

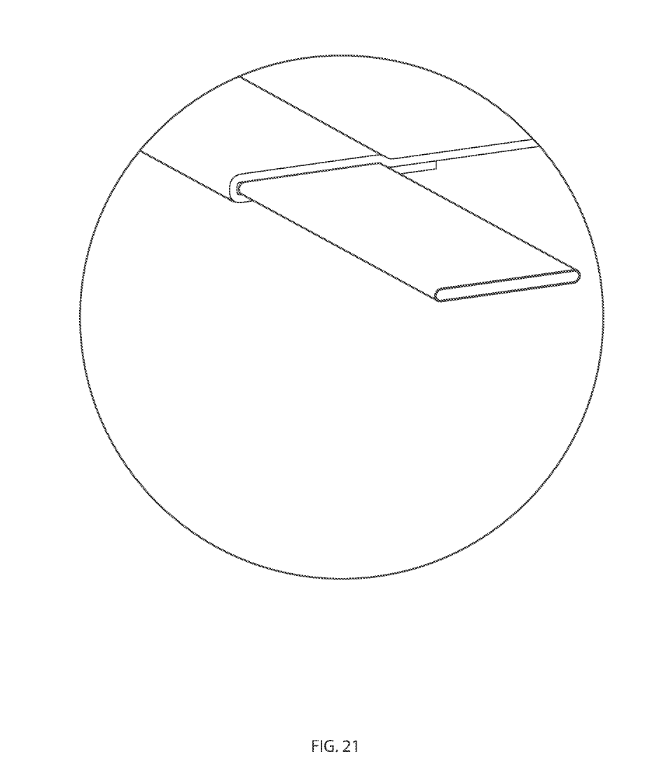

As shown in FIGS. 19-21, a support surface pad 150 can include one or more stays 155 and 160 edge stays 155, and middle stays 160, for example. The stays 155, 160 can have a level of rigidity that limit sinking and slouching of the subject into the inclinable portion 125 of the support surface 130 upon a subject laying or sitting on the support surface 130. Slouching as used herein refers to instances when, for example, the subject's neck and/or chest is flexed or bent. A subject can be slouching when, for example, the subject's shoulders and/or chest are raised a distance from the inclinable portion 125 and/or support surface pad 150 rather than resting on the inclinable portion 125 and/or support surface pad 150. Slouching can lead to an impaired airway, and upward displacement of the diaphragm, which can result in lowering the FRC of the lungs and increasing the shunt with resulting hypoxia. Sliding as used herein refers to instances when, for example, the subject moves up or down a plane of the inclinable portion 125 and/or support surface pad 150. Similar to slouching, sliding can result in the subject′ neck and/or chest being flexed or bent, thereby potentially impairing the subject's airway. The stays 155, 160 can also provide axial support to the subject's skeleton when laying or sitting on the support surface 130. The stays 155, 160, can comprise, for example, battens. The battens can be formed from various suitable materials, which can include, for example, wood as well as plastics such as acrylonitrile-butadiene-styrene and polypropylene. The battens can be die cut from a sheet material. In certain embodiments, the support surface pad 150 includes only edge stays 155 as shown in FIG. 19. In certain embodiments, the support surface pad 150 includes both middle stays 160 as well as the edge stays 155 as shown in FIGS. 20-21. The stays can vary in durometric properties as well as length and width dimensions.

Optionally, the plurality of stays 155, 160, can be inserted into the support surface pad 150 by users of the support surface pad 150. This allows end users to select and configure the level of rigidity of the support surface pad 150 to meet their respective needs. In certain embodiments, the support surface pad 150 is provided to end users preassembled, such that the plurality of stays 155, 160, or a subset, are already incorporated into the support surface pad 150.

Although the above examples describe the arrangement of loop portions and hook portions in terms of reciprocal parts, it should be understood that the arrangement can be inverted such that the plurality of loop portions are disposed in place of the plurality of hook portions, and vice versa. Loop portions and hook portions need not to be segregated, in certain embodiments for example, loop portions and hook portions can be disposed on both the outer surface 110 of the detachedly coupleable thoracic harness 100 and the inclinable portion 125 of the support surface 130 and/or the support surface pad 150.

Furthermore, although the above examples describe the fastening surface 115 of the detachedly coupleable thoracic harness 100 and the corresponding fastening surface 120 in terms of reciprocal parts of a hook-and-loop fastener system, it should be understood that the fastening surface 115 of the detachedly coupleable thoracic harness 100 and the corresponding fastening surface 120 can comprise other types of fasters, including, for example, recloseable fasteners such as Dual Lock®, sold by 3M (Saint Paul, Minn.), and other suitable fasteners include reusable adhesives, suctions, magnets, tape, and flexible magnet tape, for example.

In certain embodiments, the detachedly coupleable thoracic harness 100 includes padding material that contacts the inner surface 105. The padding material can be configured to deform upon contact with the subject's thorax in such a way so that the padding material provides a frictional force that limits translational displacement of the subject's thorax in reference to the detachedly coupleable thoracic harness 100, e.g., sliding. Padding material suitable for this purpose can include, for example, viscoelastic polyurethane foam. The padding material can help, for example, reduce sliding of the subject when the subject is releasably fastened to the inclinable portion 125 of the support surface 130. The reduction is sliding reduces the capillary pressure otherwise felt by the buttock skin of the subject, thereby reducing the likelihood of forming decubitus pressure ulcers. As indicated above, limiting sliding can also help reduce the likelihood of an impaired airway due to neck and hypoxia due to increased shunt from decreased FRC caused by chest bending.

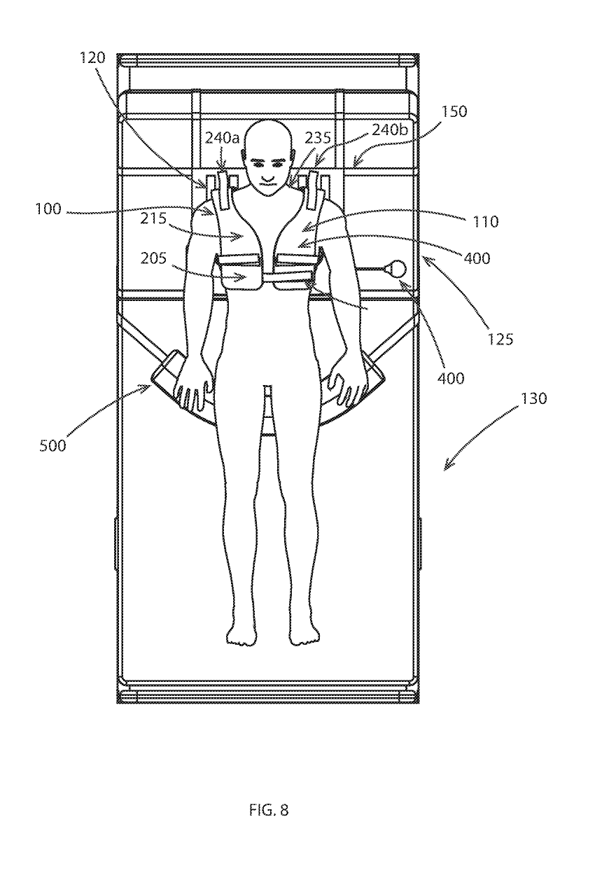

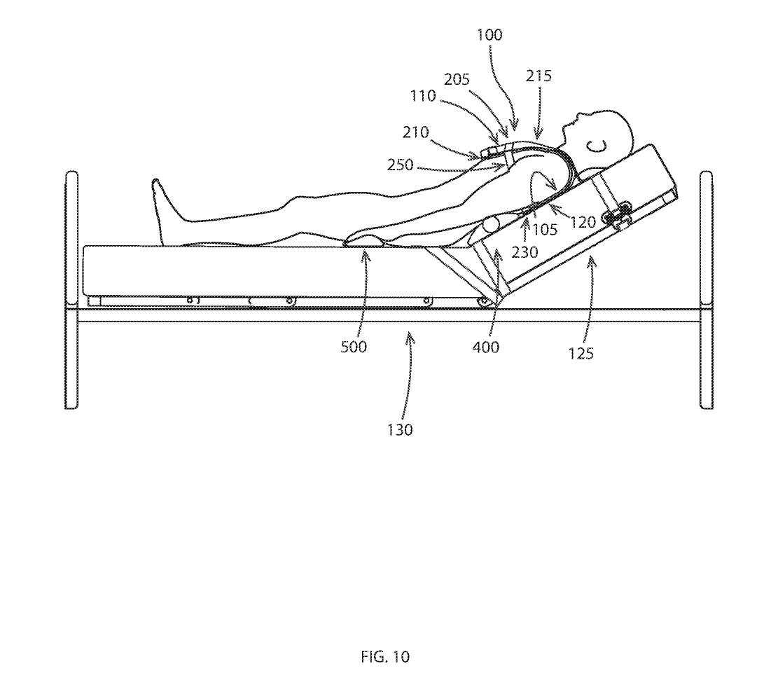

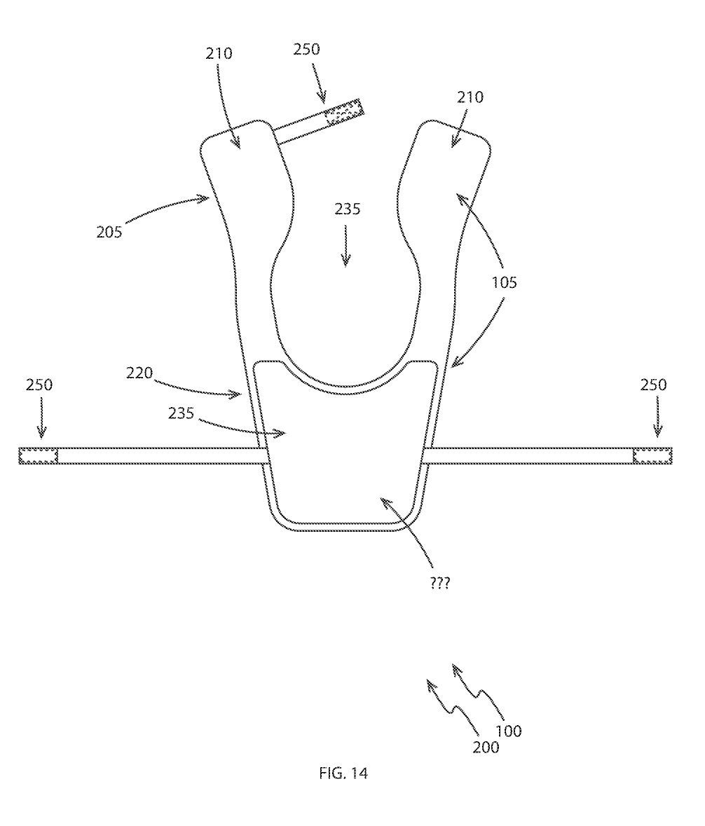

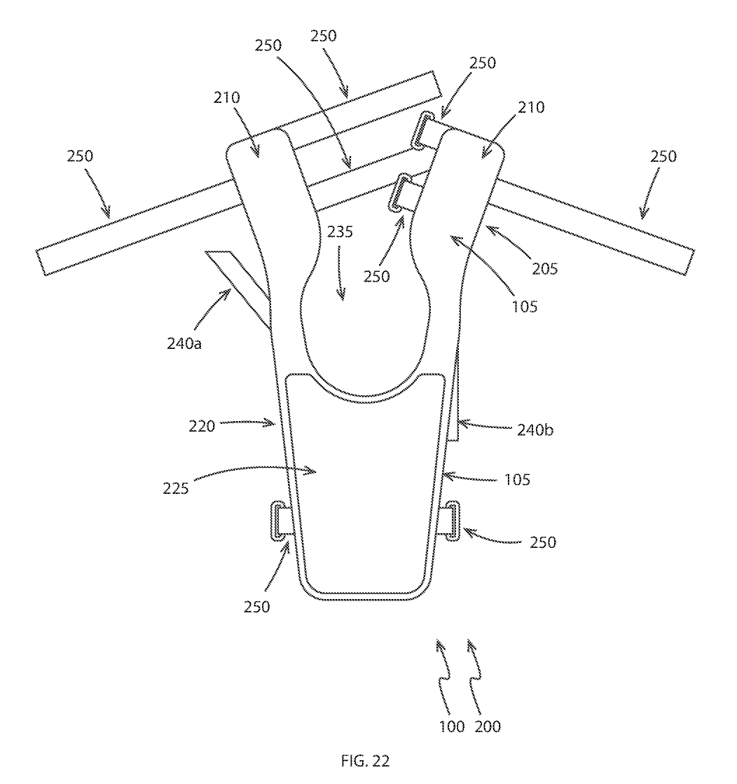

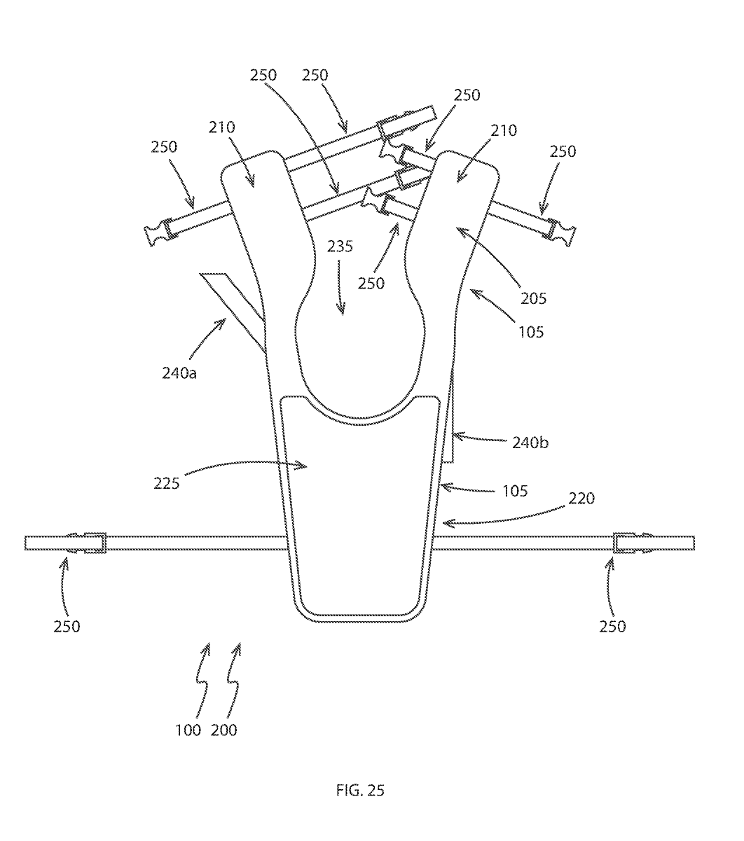

As shown in FIGS. 1, 8, 10, 13-14, and 22-25, the detachedly coupleable thoracic harness 100 can be embodied as a vest 200. An example vest 200 includes a front portion 205, a back portion 220 and an opening 235. The opening can be sized to receive at least a subject's head and/or neck. Alternatively or additionally, the opening can be sized to receive the subject's torso, arms and/or other body regions.

The front portion 205 of the vest 200 can include an inner surface 210 and an outer surface 215. The back portion 220 of the vest 200 can include an inner surface 225 and an outer surface 230. Thus, the inner surface 105 of the detachedly coupleable thoracic harness 100 can be defined by, for example, the inner surface 210 of the front portion 205 of the vest 200 and the inner surface 225 of the back portion 220 of the vest 200. Similarly the outer surface 110 of the detachedly coupleable thoracic harness 100 can be defined by, for example, the outer surface 230 of the back portion 220 of the vest 200.

Optionally, the vest 200 can include the padding material described above. The padding material can contact at least the inner surface 225 of the back portion 220 of the vest, at least the inner surface 210 of the front portion 205 of the vest, or at least both the inner surface 225 of the back portion 220 of the vest 210 and the inner surface 210 of the front portion 205 of the vest 200. The padding material can limit translational displacement of the subject's upper body in reference to vest 200, and the vest 200 can limit sliding of the subject when the inclinable portion 125 is inclined at an angle relative to another portion of the support surface 130, such as a foot portion of a folding bed. The padding material can help prevent the vest 200 from sliding into the subject's brachial plexus.

Optionally, the front portion 205 of the vest 200 can include semi-rigid stays that limit anterior bend of the subject's chest, thus allowing for improved movement of the diaphragm during breathing. Optionally, the back portion 220 of the vest 200 can include flexible stays. The flexible stays can enhance the contact area between the fastening surface 115 and the corresponding fastening surface 120 by, for example, distributing the weight of the subject throughout the outer surface back portion 220 of the vest 200. Thus in certain embodiments, the entire outer surface 230 of the back portion 220 of vest 200 is mated to the inclinable portion 125 of the support surface 130 when releasably coupled.

As shown in FIGS. 1, 8, 13, 22-25, the vest 200 can optionally include one or more shoulder straps 240a, 240b, which can anchor one or more anterior shoulders of the subject to the inclinable portion 125 of the support surface 130 and/or the support surface pad 150. Similar to the fastening surface 115 and corresponding fastening surface 120, the one or more shoulder straps 240a, 240b can be anchored to the inclinable portion 125 and/or the support surface pad 150 using various suitable fasteners, such the ones disclosed above. The use of the shoulder straps 240a, 240b in combination with the fastening surfaces 115 and 120, provide an additional fastening surface to further limit the sliding and slouching of the subject on the inclinable portion 125.

As shown in FIGS. 1, 8, 10, 13-14, and 22-25 the vest 200 can optionally include one or more vest straps 250 with fasteners that releasably secure the front portion 205 of the vest 200 to the back portion 220 of the vest 200. The Vest straps 250 can include fasteners, such as clasps, buckles, and the other suitable fasteners, like the ones referenced herein.

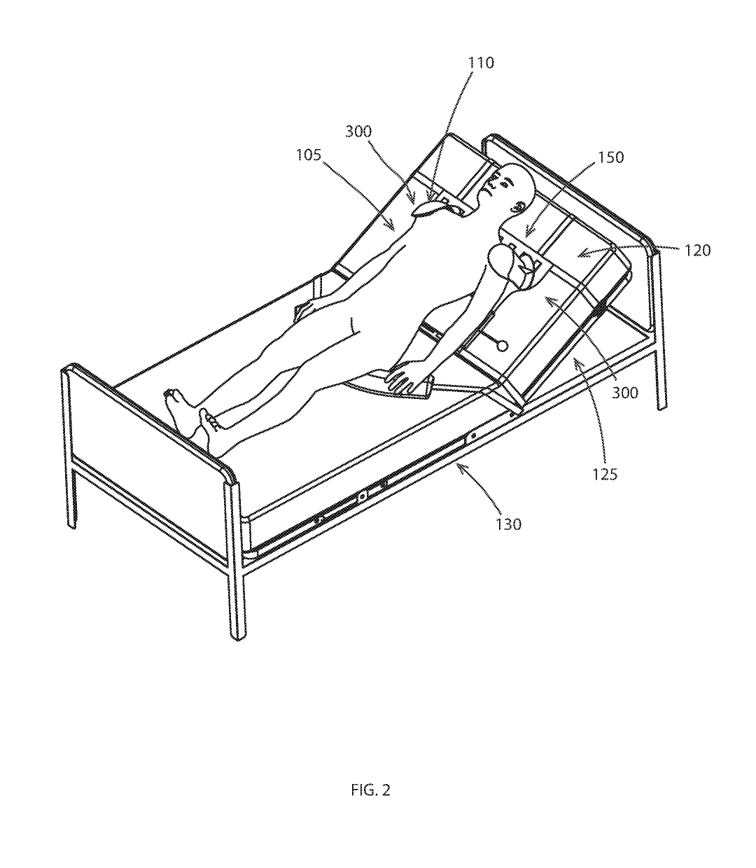

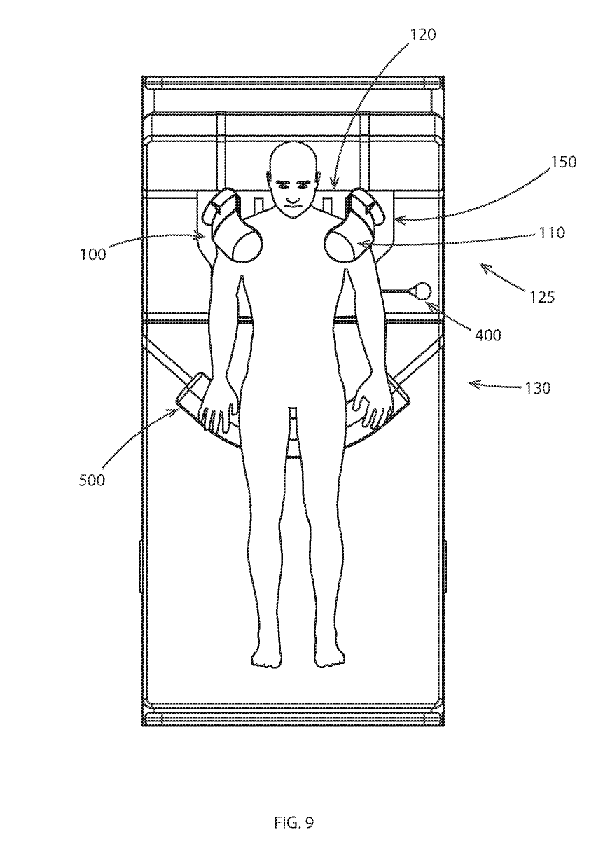

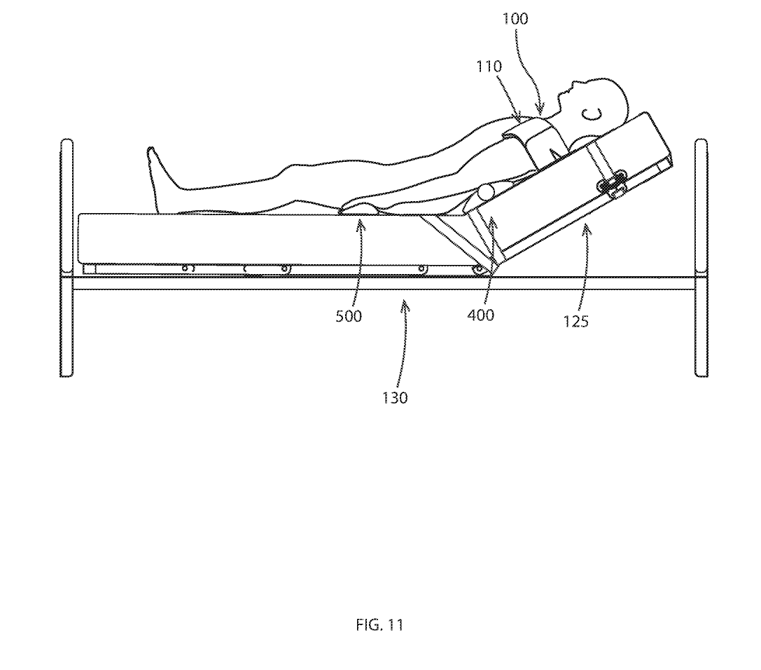

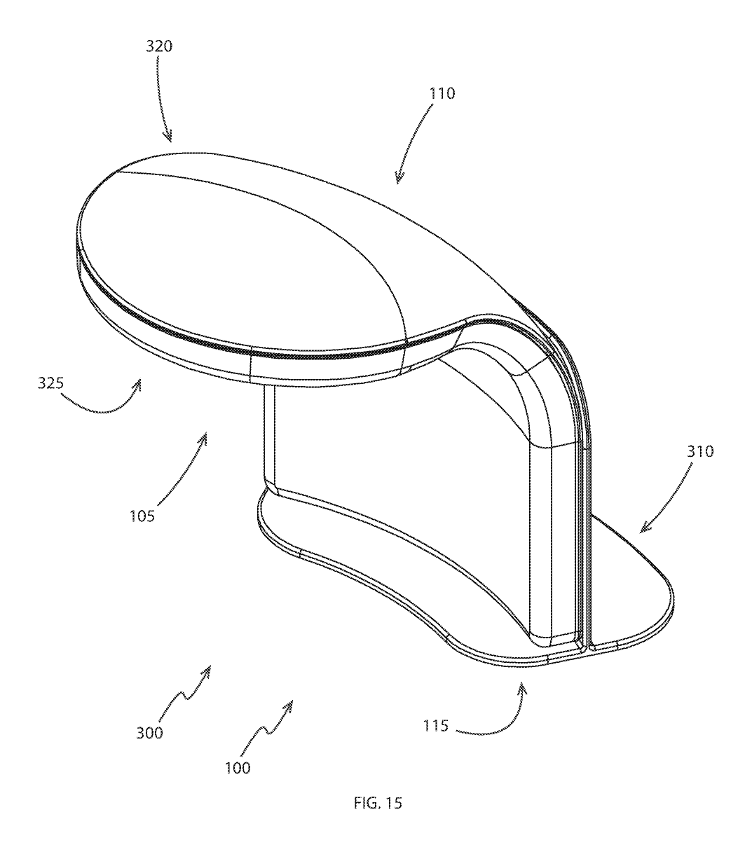

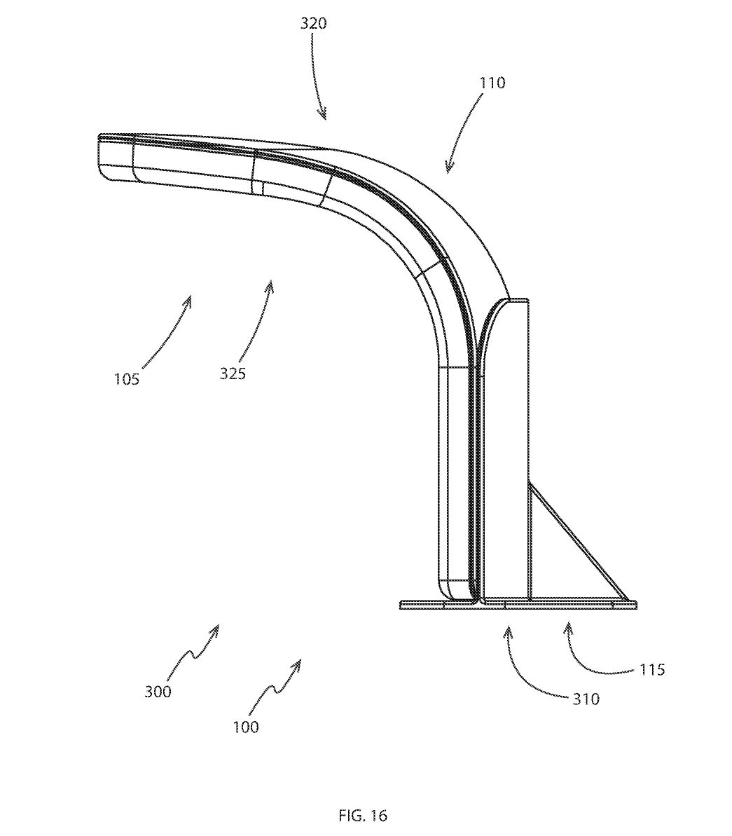

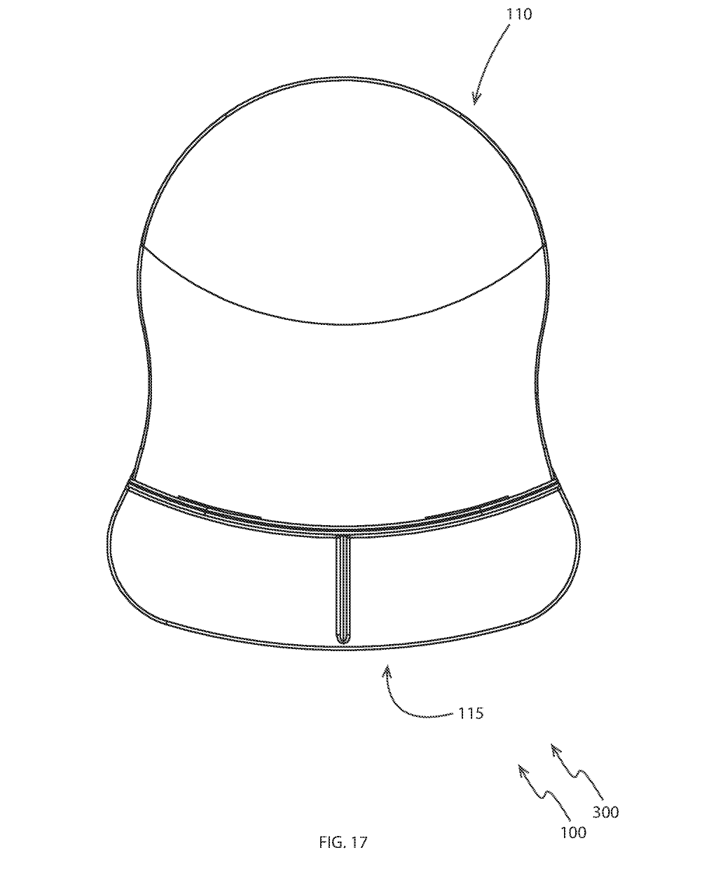

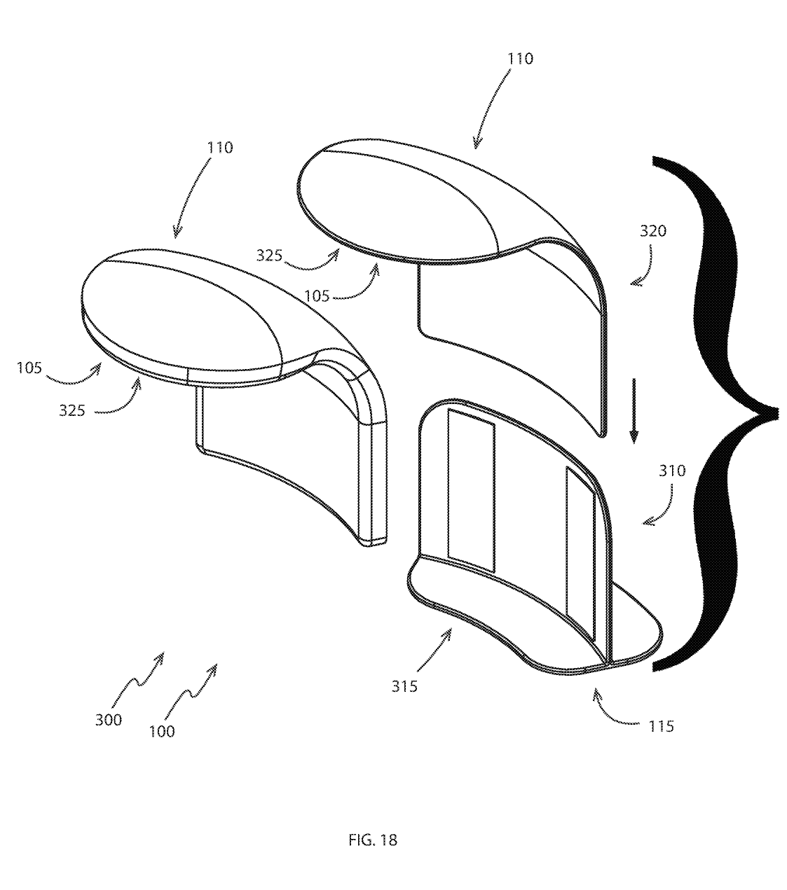

As shown in FIGS. 2, 9, 11, 15-18, in certain embodiments the detachedly coupleable thoracic harness 100 can be embodied as one or more shoulder guards 300. An example shoulder guard 300 can include a base portion 310 and a cuff portion 320. The base portion 310 can have an outer surface 310 which can define the outer surface 110 of the detachedly coupleable thoracic harness 100. The cuff portion 320 can reversibly interlock with the base portion 310. The cuff portion 320 can further include an inner surface 325, which can define the inner surface 105 of the detachedly coupleable thoracic harness 100. Similar to the vest 200, the padding material described above can contact the inner surface 105 of the cuff portion 320 and be used to limit translational displacement of the subject's thorax in reference to the pair of shoulder guards 300. In certain embodiments, the shoulder guards 300 can comprise a pair of shoulder guards as shown in FIGS. 2, 9, 11, 15-18. In certain embodiments the pair of shoulder guards 300 can share a common base portion.

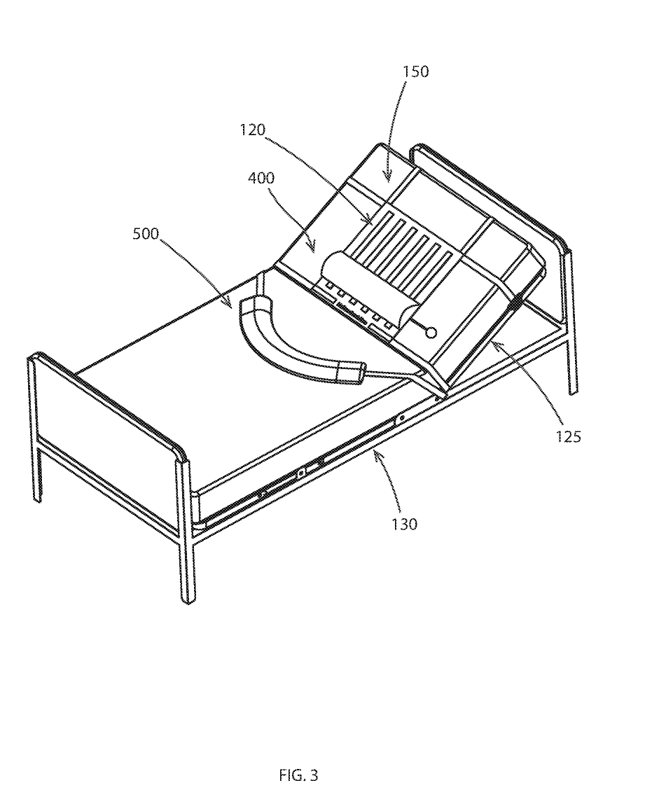

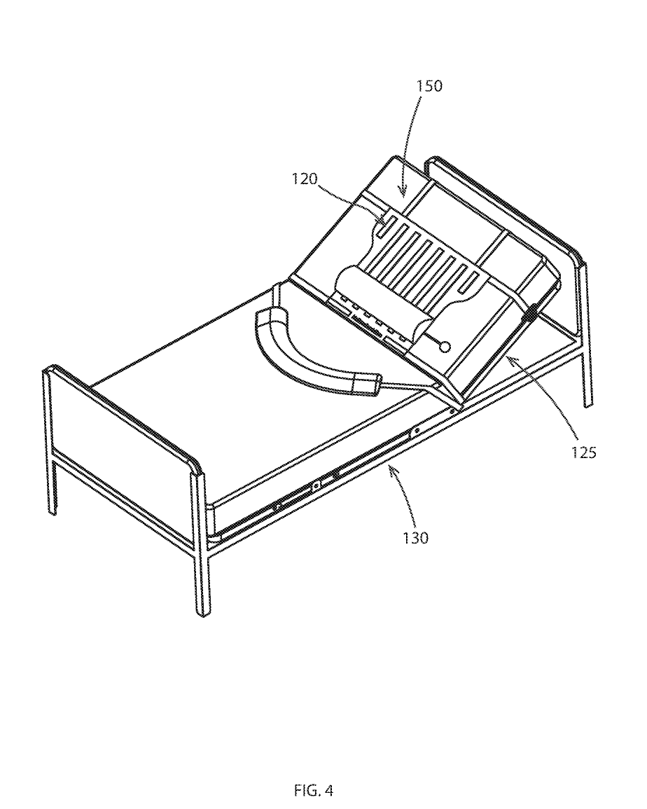

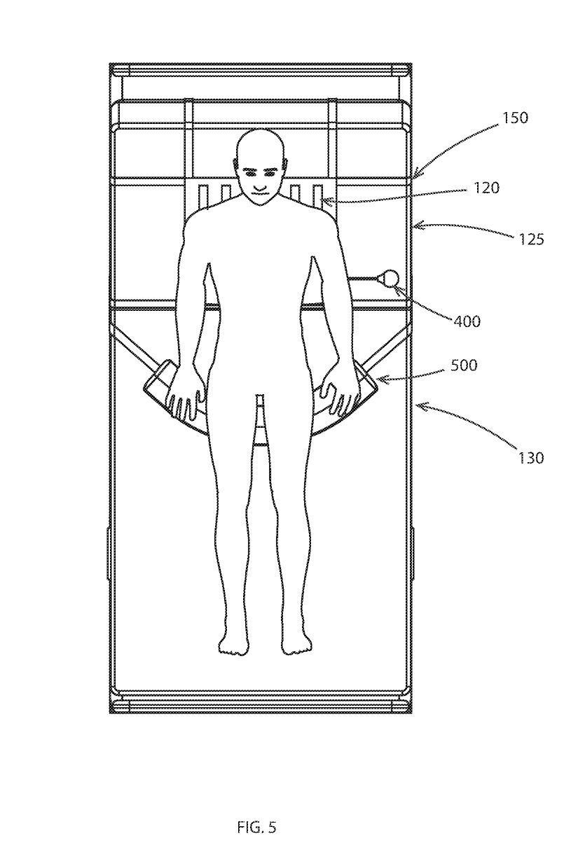

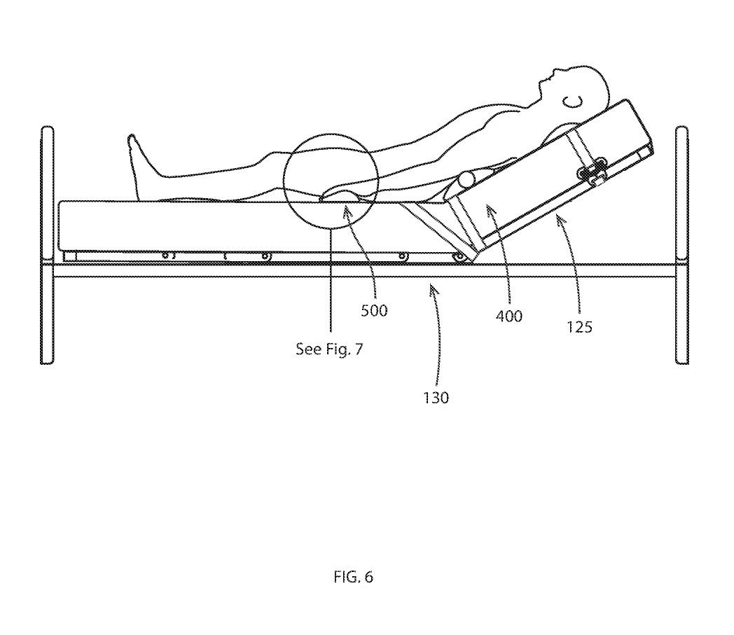

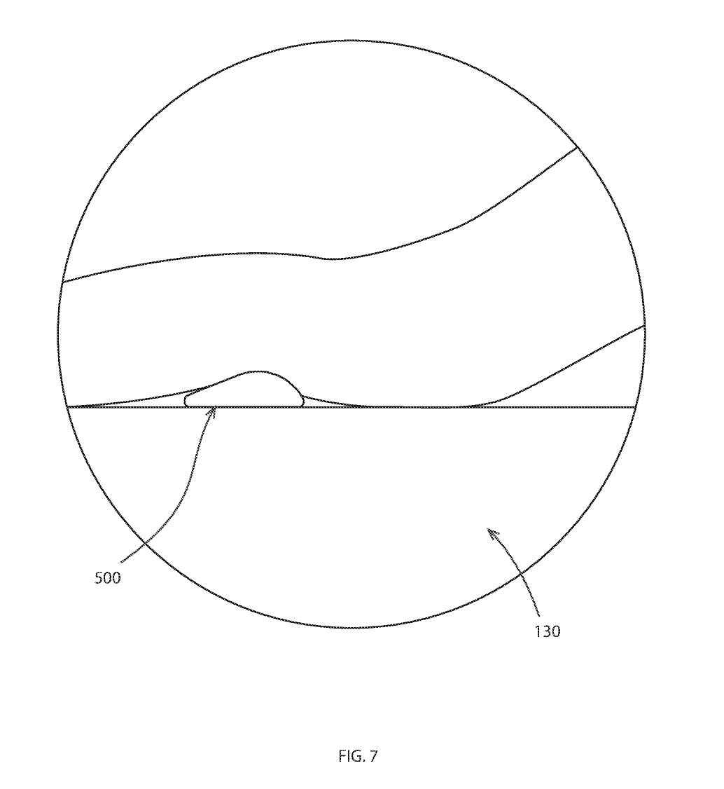

As shown in FIGS. 1-11, an example system for effecting posture can include, additionally or alternatively, a lumbar support 400 that can be attached to a lower portion of the inclinable portion 125 of the support surface. Similar to the example detachedly coupleable thoracic harness 100 described above, the lumbar support 400 can include padding material, such as viscoelastic polyurethane foam, which can be configured to deform upon the subject's lower back making contact with the lumbar support 400. The lumbar support 400 can limit slouching and sliding of the subject when the lumbar support 400 makes contact with the subject by, for example, exaggerating the lumbar S-Shaped configuration of the subject's spine. Exaggerating the lumbar S-Shape configuration in the manner disclosed herein can help promote lordosis and maximize contact of the detachedly coupleable thoracic harness 100 to the inclinable portion 125 of the support surface 130. In some embodiments, the lumbar support 500 can be inflatable, either through automatic or manual means. In certain embodiments, the lumbar support can be automatically inflated such that an angle between the subject's back and his/her legs is adjusted in response to an occurrence of a predetermined event, such as at a predetermined time period or upon detecting that the HOB is presently below a designed angle.

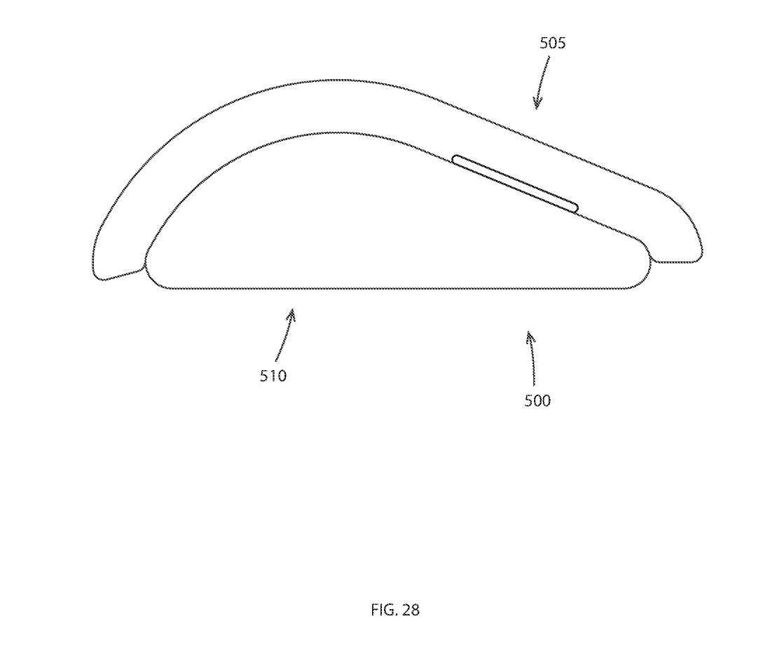

As shown in FIGS. 1-11 and 26-29, an example system for effecting posture can additionally or alternatively include a femoral-gluteal pad 500 that is attachable to, for example, another portion of the support surface 130. The femoral-gluteal pad can include padding material, similar to the ones described above, configured to deform upon the subject's femoral and or gluteal body region making contact with the femoral gluteal pad 500. The femoral-gluteal pad 500 can provide a frictional force that limits translational displacement of the subject's femoral and/or gluteal body region from the support surface 125. As provided above, the padding material can include, for example, viscoelastic polyurethane foam. In certain embodiments the femoral-gluteal pad can include a cutout, such as a coccyx cutout. The femoral-gluteal pad can help reduce the amount of capillary pressure otherwise felt by the buttock skin of the subject, thereby reducing the likelihood of forming decubitus pressure ulcers.

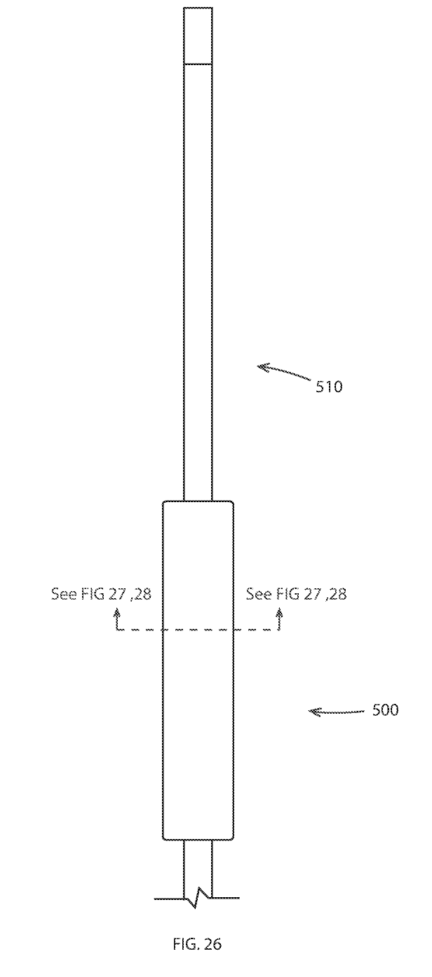

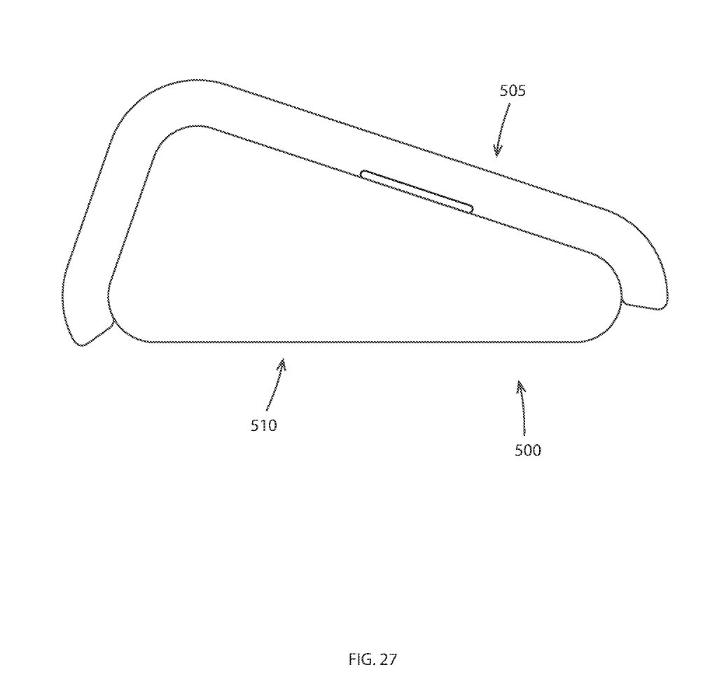

As shown in FIGS. 26-27, the padding material can include several layers, for example, an open cell low density foam layer 505 and a closed cell high density foam layer 510. The femoral-gluteal pad 500 can be coupled to the support surface 130 using, for example, a strap 520. The strap 520 can be situated between the open cell low density foam layer 505 and the closed cell high density foam layer 510, for example.

Also provided are methods for effecting posture of a subject. The methods can be used, for example, to effect posture of a subject who is to be positioned on as support surface having an inclinable portion such that the subject thorax is inclinable relative to the subject's legs. An example method includes providing a detachedly coupleable thoracic harness, wherein the detachedly coupleable thoracic harness includes an inner surface contoured to fit over the shoulders of a subject and includes an outer surface having a fastening surface that releasably attaches to a corresponding fastening surface disposed on an inclinable portion of a support surface. The method further includes placing the detachedly coupleable thoracic harness over the shoulders of the subject. The method further includes coupling the detachedly coupleable thoracic harness to the inclinable portion of the support surface by at least releasably attaching the fastening surface of the detachedly coupleable thoracic harness to the corresponding fastening surface disposed on the inclinable portion of the support surface. Optionally, the method further includes detaching the detachedly coupleable thoracic harness from the inclinable portion of the support surface.

Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention, which is defined in the following claims and all equivalents thereto. Further, it is recognized that many embodiments may be conceived that do not achieve all of the advantages of some embodiments, yet the absence of a particular advantage shall not be construed to necessarily mean that such an embodiment is outside the scope of the present invention.

Disclosed are materials, systems, devices, compositions, and components that can be used for, can be used in conjunction with, can be used in preparation for, or are products of the disclosed methods, systems and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein.

### Embodiments

The following is a non-exclusive list of exemplary embodiments in accordance with the present disclosure:

1. A system for effecting posture of a subject who is to be positioned on a support surface having an inclinable portion such that the subject's thorax is inclinable relative to the subject's legs, the system comprising:
a detachedly coupleable thoracic harness having at least:
   an inner surface contoured to fit over shoulders of the subject; and
   an outer surface having a fastening surface that releasably attaches to a corresponding fastening surface disposed on the inclinable portion of the support surface.

2. The system of embodiment 1, wherein the detachedly coupleable thoracic harness further includes a padding material that contacts the inner surface of the detachedly coupleable thoracic harness, wherein the padding material is configured to deform upon contact with the subject's thorax in such a way so that the padding material provides a frictional force that limits translational displacement of the subject's thorax in reference to the detachedly coupleable thoracic harness.

3. The system of embodiment 2, wherein the padding material includes viscoelastic polyurethane foam.

4. The system of embodiment 1, wherein the fastening surface and the corresponding fastening surface comprise reciprocal parts of a hook-and-loop fastener system, wherein the hook-and-loop fastener system includes a plurality of hook portions and a plurality of loop portions.

5. The system of embodiment 4, wherein the plurality of hook portions are disposed on the outer surface of the detachedly coupleable thoracic harness, and wherein the plurality of loop portions are disposed on the inclinable portion of the support surface to releasably fasten the outer surface of the detachedly coupleable thoracic harness to the inclinable portion of the support surface.

6. The system of embodiment 5, wherein the plurality of hook portions are disposed symmetrically down the outer surface of the detachedly coupleable thoracic harness

7. The system of embodiment 6, wherein the plurality of hook portions are disposed symmetrically down the outer surface of the detachedly coupleable thoracic harness as a plurality of patches.

8. The system of embodiment 7, wherein a subset of the plurality of patches have a circular shape.

9. The system of embodiment 7, wherein a subset of the plurality of patches have a triangular shape.

10. The system of embodiment 5, wherein the plurality of loop portions are disposed on a support surface pad coupled to the support surface.

11. The system of embodiment 10, wherein the support surface pad includes a plurality of stays having a level of rigidity to limit sinking and slouching of the subject into the inclinable portion of the support surface when laying or sitting on the support surface, wherein the plurality of stays provide axial support of the subject's skeleton when laying or sitting on the support surface.

12. The system of embodiment 5, wherein the plurality of loop portions are disposed as a plurality of parallel strips along the inclinable portion of the support surface.

13. The system of embodiment 4, wherein the plurality of loop portions are disposed on the outer surface of the detachedly coupleable thoracic harness, and wherein the plurality of hook portions are disposed on the inclinable portion of the support surface to releasably fasten the outer surface of the detachedly coupleable thoracic harness to the inclinable portion of the support surface.

14. The system of embodiment 13, wherein the plurality of hook portions are disposed as a plurality of parallel strips along the inclinable portion of the support surface.

15. The system of embodiment 14, wherein the plurality of loop portions are disposed symmetrically down the outer surface of the detachedly coupleable thoracic harness.

16. The system of embodiment 15, wherein the plurality of hook portions are disposed symmetrically down the outer surface of the detachedly coupleable thoracic harness as a plurality of patches.

17. The system of embodiment 16, wherein a subset of the plurality of patches have a circular shape.

18. The system of embodiment 16, wherein a subset of the plurality of patches have a triangular shape.

19. The system of embodiment 14, wherein the plurality of hook portions are disposed on a support surface pad coupled to the support surface.

20. The system of embodiment 19, wherein the support surface pad includes a plurality of stays having a level of rigidity to limit sinking of the subject into the inclinable portion of the support surface when laying or sitting on the support surface, wherein the plurality of stays provide axial support of the subject's skeleton when laying or sitting on the support surface.

21. The system of any of embodiments 1-20, wherein the detachedly coupleable thoracic harness comprises a vest having at least:
a front portion comprising an inner surface and an outer surface;
a back portion comprising an inner surface and an outer surface;
an opening for receiving at least a subject's head and neck;
wherein the inner surface of the detachedly coupleable thoracic harness is defined by the inner surface of the front portion and the inner surface of the back portion of the vest; and
wherein the outer surface of the detachedly coupleable thoracic harness is defined by the outer surface of the back portion of the vest.

22. The system of embodiment 21, wherein the padding material contacts the inner surface of the back portion of the vest.

23. The system of embodiment 21, wherein the padding material contacts the inner surface of the front portion of the vest.

24. The system of embodiment 21, wherein the padding material contacts the inner surface of the back portion of the vest and the inner surface of the front portion of the vest.

25. The system of any of embodiments 21-24, wherein the front portion of the vest includes semi-rigid stays that limit anterior bend of the subject's chest.

26. The system of any of embodiments 21-25, wherein the vest further includes a shoulder strap that anchors an anterior shoulder of the subject to the inclinable portion of the support surface.

27. The system of any of embodiments 21-25, wherein the vest further includes:
a first shoulder strap that anchors a first anterior shoulder of the subject to the inclinable portion of the support surface; and
a second shoulder strap that anchors a second anterior shoulder of the subject to the inclinable portion of the support surface.

28. The system of any of embodiments 1-20, wherein the detachedly coupleable thoracic harness comprises a pair of shoulder guards.

29. The system of embodiment 28, wherein each of the pair of shoulder guards includes:
a base portion, wherein the outer surface of the detachedly coupleable thoracic harness is defined by an outer surface of the base portion; and
a cuff portion that reversibly interlocks with the base portion, wherein the inner surface of the detachedly coupleable thoracic harness is defined by an inner surface of the cuff portion.

30. The system of embodiment 28, wherein the pair of shoulder guards share a common base portion.

31. The system of any of embodiments 29-30, wherein the padding material contacts the inner surface of the cuff portion.

32. The system of any of embodiments 1-31, further comprising a lumbar support attachable to a lower portion of the inclinable portion of the support surface, wherein the lumbar support limits slouching and sliding of the subject when the lumbar support makes contact with the subject.

33. The system of embodiment 32, wherein the lumbar support limits slouching and sliding of the subject by at least exaggerating lumbar S-shaped configured of the subject's spine to promote lordosis and to maximize contact of the detachedly coupleable thoracic harness to the inclinable portion of the support surface.

34. The system of any of embodiments 32-33, wherein the lumbar support is inflatable.

35. The system of any of embodiments 32-34, wherein the lumbar support includes viscoelastic polyurethane foam.

36. The system of any of embodiments 1-35, further comprising a femoral-gluteal pad that is attachable to another portion of the support surface.

37. The system of embodiment 36, wherein the femoral-gluteal pad includes a second padding material that is configured to deform upon the subject's femoral and/or gluteal body region making contact with the femoral-gluteal pad, wherein the femoral-gluteal pad provides a frictional force that limits translational displacement of the subject's femoral and/or gluteal body region in reference to the femoral-gluteal pad.

38. The system of any of embodiments 36-37, wherein the second padding material includes viscoelastic polyurethane foam.

39. The system of any of embodiments 36-38, wherein the femoral-gluteal pad includes a coccyx cutout.

40. A method for effecting posture of a subject who is to be positioned on a support surface having an inclinable portion such that the subject's thorax is inclinable relative to the subject's legs, the method comprising:
providing a detachedly coupleable thoracic harness, wherein the detachedly coupleable thoracic harness includes an inner surface contoured to fit over shoulders of the subject and includes an outer surface having a fastening surface that releasably attaches to a corresponding fastening surface disposed on the inclinable portion of the support surface;
placing the detachedly coupleable thoracic harness over the shoulders of the subject; and
coupling the detachedly coupleable thoracic harness to the inclinable portion of the support surface by at least releasably attaching the fastening surface of the detachedly coupleable thoracic harness to the corresponding fastening surface disposed on the inclinable portion of the support surface.

41. The method of embodiment 40, further comprising detaching the detachedly coupleable thoracic harness from the inclinable portion of the support surface.

42. A system for effecting posture of a subject who is to be positioned on a support surface having an inclinable portion such that the subject's thorax is inclinable relative to the subject's legs, the system comprising:
a vest comprising:
   a front portion comprising an inner surface and an outer surface;
   a back portion comprising an inner surface and an outer surface, wherein the outer surface of the back portion includes a fastening surface that releasably attaches to a corresponding fastening surface disposed on the inclinable portion of the support surface;
   an opening for receiving at least a subject's head and neck; and
   a padding material that contacts the inner surface of the front portion, wherein the padding material is configured to deform upon contact with the subject's thorax in such a way so that the padding material provides a frictional force that limits translational displacement of the subject's thorax in reference to vest;
an inflatable lumbar support that is attachable to a lower portion of the inclinable portion of the support surface, wherein the inflatable lumbar supports limits slouching and sliding of the subject upon the lumbar support making contact with the subject; and
a femoral-gluteal pad that is attachable to another portion of the support surface, wherein the femoral-gluteal pad includes a second padding material that is configured to deform upon the subject's femoral and/or gluteal body region making contact with the femoral-gluteal pad, wherein the femoral-gluteal pad provides a frictional force that limits translational displacement of the subject's femoral and/or gluteal body region in reference to the femoral-gluteal pad.

43. A system for effecting posture of a subject who is to be positioned on a support surface having an inclinable portion such that the subject's thorax is inclinable relative to the subject's legs, the system comprising:
a pair of shoulder guards each comprising:
   a cuff portion contoured to fit over shoulders of the subject;
   a base portion having a fastening surface that releasably attaches to a corresponding fastening surface disposed on the inclinable portion of the support surface;
   a padding material that contacts an inner surface of the cuff portion, wherein the padding material is configured to deform upon contact with the subject's thorax in such a way so that the padding material provides a frictional force that limits translational displacement of the subject's shoulders in reference to the cuff portion;
an inflatable lumbar support that is attachable to a lower portion of the inclinable portion of the support surface, wherein the inflatable lumbar supports limits slouching and sliding of the subject upon the lumbar support making contact with the subject; and
a femoral-gluteal pad that is attachable to another portion of the support surface, wherein the femoral-gluteal pad includes a second padding material that is configured to deform upon the subject's femoral and/or gluteal body region making contact with the femoral-gluteal pad, wherein the femoral-gluteal pad provides a frictional force that limits translational displacement of the subject's femoral and/or gluteal body region in reference to the femoral-gluteal pad.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. A method for effecting posture of a subject who is to be positioned on a support surface having an inclinable portion such that the subject's thorax is inclinable relative to the subject's legs, the method comprising:
providing a detachedly coupleable thoracic harness, wherein the detachedly coupleable thoracic harness includes an inner surface contoured to fit over shoulders of the subject and includes an outer surface having a fastening surface that releasably attaches to a corresponding fastening surface disposed on the inclinable portion of the support surface;
placing the detachedly coupleable thoracic harness over the shoulders of the subject; and
coupling the detachedly coupleable thoracic harness to the inclinable portion of the support surface by at least releasably attaching the fastening surface of the detachedly coupleable thoracic harness to the corresponding fastening surface disposed on the inclinable portion of the support surface.

29. A system for effecting posture of a subject who is to be positioned on a support surface having an inclinable portion such that the subject's thorax is inclinable relative to the subject's legs, the system comprising:
a vest comprising:
a front portion comprising an inner surface and an outer surface;
a back portion comprising an inner surface and an outer surface, wherein the outer surface of the back portion includes a fastening surface that releasably attaches to a corresponding fastening surface disposed on the inclinable portion of the support surface;
a first shoulder strap coupled to the back portion of the vest, wherein the first should strap is configured to anchor a first anterior shoulder of the subject to the inclinable portion of the support surface;
a second shoulder strap coupled to the back portion of the vest, wherein the second shoulder strap is configured to anchor a second anterior shoulder of the subject to the inclinable portion of the support surface;
an opening for receiving at least a subject's head and neck; and
a padding material that contacts the inner surface of the front portion, wherein the padding material is configured to deform upon contact with the subject's thorax in such a way so that the padding material provides a frictional force that limits translational displacement of the subject's thorax in reference to vest.

30. A system for effecting posture of a subject who is to be positioned on a support surface having an inclinable portion such that the subject's thorax is inclinable relative to the subject's legs, the system comprising:
a pair of separate shoulder guards each comprising:
a cuff portion contoured to fit over a respective shoulder of the subject;
a base portion having a fastening surface that releasably attaches to a corresponding fastening surface disposed on the inclinable portion of the support surface, wherein the cuff portion reversibly interlocks with the base portion; and
a padding material that contacts an inner surface of the cuff portion, wherein the padding material is configured to deform upon contact with the subject's thorax in such a way so that the padding material provides a frictional force that limits translational displacement of the subject's shoulders in reference to the cuff portion.

31. The system of claim 29, wherein the padding material includes viscoelastic polyurethane foam.

32. The system of claim 31, wherein the front portion of the vest includes semi-rigid stays that limit anterior bend of the subject's chest.

33. The system of claim 32, wherein the fastening surface and the corresponding fastening surface comprise reciprocal parts of a hook-and-loop fastener system, wherein the hook-and-loop fastener system includes a plurality of hook portions and a plurality of loop portions.

34. The system of claim 33, wherein the plurality of hook portions are disposed on the outer surface of the of the back portion of the vest as a plurality of horizontal strips, and wherein the plurality of loop portions are disposed on the inclinable portion of the support surface to releasable fasten the outer surface of the back portion of the vest to the inclinable portion of the support surface.

35. The system of claim 34, wherein the plurality of loop portions are disposed as a plurality of parallel strips along the inclinable portion of the support surface.

36. The system of claim 34, wherein the plurality of loop portions are disposed on a support surface pad coupled to the support surface.

37. The system of claim 36, wherein the support surface pad includes a plurality of stays, wherein the plurality of stays provide axial support of the subject's skeleton when laying or sitting on the support surface.

38. The system of claim 33, wherein the plurality of loop portions are disposed on the outer surface of the back portion of the vest as a plurality of horizontal strips, and wherein the plurality of hook portions are disposed on the inclinable portion of the support surface to releasably fasten the outer surface of the back portion of the vest to the inclinable portion of the support surface.

39. The system of claim 38, wherein the plurality of loop portions are disposed as a plurality of parallel strips along the inclinable portion of the support surface.

40. The system of claim 38, wherein the plurality of hook portions are disposed on a support surface pad coupled to the support surface.

41. The system of claim 40, wherein the support surface pad includes a plurality of stays, wherein the plurality of stays provide axial support of the subject's skeleton when laying or sitting on the support surface.

42. The system of claim 32 further comprising an inflatable lumbar support that is attachable to a lower portion of the inclinable portion of the support surface, wherein the inflatable lumbar support limits slouching and sliding of the subject upon the lumbar support making contact with the subject.

43. The system of claim 42 further comprising a femoral-gluteal pad that is attachable to another portion of the support surface, wherein the femoral-gluteal pad includes a second padding material that is configured to deform upon the subject's femoral and/or gluteal body region making contact with the femoral-gluteal pad, wherein the femoral-gluteal pad provides a frictional force that limits translational displacement of the subject's femoral and/or gluteal body region in reference to the femoral-gluteal pad.

44. The system of claim 30, wherein the padding material includes viscoelastic polyurethane foam.

45. The system of claim 44, wherein the fastening surface and the corresponding fastening surface comprise reciprocal parts of a hook-and-loop fastener system, wherein the hook-and-loop fastener system includes a plurality of hook portions and a plurality of loop portions.

46. The system of claim 45, wherein the plurality of hook portions are disposed on an outer surface of each of the base portions of the pair of separate shoulder guards, the hook portions being disposed as a plurality of horizontal strips, and wherein the plurality of loop portions are disposed on the inclinable portion of the support surface to releasably fasten the base portions of the pair of separate shoulder guards to the inclinable portion of the support surface.

47. The system of claim 46, wherein the plurality of loop portions are disposed as a plurality of parallel strips along the inclinable portion of the support surface.

48. The system of claim 46, wherein the plurality of loop portions are disposed on a support surface pad coupled to the support surface.

49. The system of claim 48, wherein the support surface pad includes a plurality of stays, wherein the plurality of stays provide axial support of the subject's skeleton when laying or sitting on the support surface.

50. The system of claim 45, wherein the plurality of loop portions are disposed on an outer surface of each of the base portions of the pair of separate shoulder guards, the loop portions being disposed as a plurality of horizontal strips, and wherein the plurality of hook portions are disposed on the inclinable portion of the support surface to releasably fasten the base portions of the pair of separate shoulder guards to the inclinable portion of the support surface.

51. The system of claim 50, wherein the plurality of loop portions are disposed as a plurality of parallel strips along the inclinable portion of the support surface.

52. The system of claim 50, wherein the plurality of hook portions are disposed on a support surface pad coupled to the support surface.

53. The system of claim 52, wherein the support surface pad includes a plurality of stays, wherein the plurality of stays provide axial support of the subject's skeleton when laying or sitting on the support surface.

54. The system of claim 44 further comprising an inflatable lumbar support that is attachable to a lower portion of the inclinable portion of the support surface, wherein the inflatable lumbar support limits slouching and sliding of the subject upon the lumbar support making contact with the subject.

55. The system of claim 54 further comprising a femoral-gluteal pad that is attachable to another portion of the support surface, wherein the femoral-gluteal pad includes a second padding material that is configured to deform upon the subject's femoral and/or gluteal body region making contact with the femoral-gluteal pad, wherein the femoral-gluteal pad provides a frictional force that limits translational displacement of the subject's femoral and/or gluteal body region in reference to the femoral-gluteal pad.
